(19) United States
(12) Patent Application Publication
(10) Pub. No.: US 2005/0239045 A1
(43) Pub. Date: Oct. 27, 2005

(54) Microorganism or cell collecting method, and microorganism or cell collecting implement used for the method

(75) Inventors: Masashi Okamoto, Kyoto-shi (JP); Tatsuo Kamata, Kyoto-shi (JP); Yuji Izumizawa, Kyoto-shi (JP); Atsushi Murakami, Kyoto-shi (JP)

Correspondence Address: MERCHANT & GOULD PC, P.O. BOX 2903, MINNEAPOLIS, MN, 55402-0903, US

(73) Assignee: ARKRAY Inc., Kyoto (JP)

(21) Appl. No.: 10/522,045

(22) PCT Filed: Dec. 8, 2003
(86) PCT No.: PCT/JP03/15686

§ 371 Date: Jan. 19, 2005

(51) Int. Cl. C12Q 1/00; C12N 1/20

(52) U.S. Cl. 435/4; 435/252.1; 435/325

(57) ABSTRACT

A method capable of easily collecting microorganisms or cells in a short time without using a special device, comprising the steps of preparing a centrifugal tube (1) in which the inside thereof is divided into upper and lower parts by a filter (14) and water absorbing resin particles (15) are disposed on the filter (14), pouring liquid specimen in the centrifugal tube (1) so that the liquid specimen touches the water absorbing resin particles (15) to absorb the liquid specimen by the resin particles so as to arrest microorganisms or cells on the surfaces of the particles, pouring collecting liquid into the centrifugal tube (1) to collect the microorganisms or cells with the collecting liquid and, by centrifugal separation, accumulating the collecting liquid at the bottom part of the centrifugal tube (1) through the filter (14).

### TECHNICAL FIELD

The present invention relates to a method of collecting microorganisms or cells from a liquid sample and to a method of performing gene amplification or gene detection using the same.

### BACKGROUND ART

Tuberculosis still is a serious bacterial disease worldwide, and not only treatment methods but also diagnostic methods therefor are extremely important. A final diagnosis as to the tuberculosis infection is made by carrying out a culture method. However, since bacteria responsible for tuberculosis (hereinafter referred to as “TB bacteria”) have an extremely slow growth rate, the establishment of a preliminary diagnostic method to be performed prior to the culture method has been desired. As such a preliminary diagnostic method, a genetic analysis method using a polymerase chain reaction (PCR) method is attracting attention. In this method, a primer specific to a gene of TB bacteria is used to amplify a gene of the TB bacteria, if any, so that it can be detected, thereby enabling the presence or absence of the TB bacteria to be determined.

In the above-described preliminary diagnostic method using the PCR method, collecting TB bacteria from a liquid sample such as sputum is necessary as a pretreatment. Conventionally, the collection of the TB bacteria has been carried out in the following manner, for example (“New Guideline for TB Bacteria Test 2000” published by Japan Anti-Tuberculosis Association, pp. 28-29). First, a solution (a NALC-NaOH solution) containing N-acetyl-L-cysteine (NALC) and sodium hydroxide (NaOH) is added to the liquid sample to remove the viscosity therefrom, thus preparing a sample solution. The sample solution is centrifuged at 13000 g for 10 minutes, and the precipitated TB bacteria are collected. However, the conventional collection method using centrifugation has problems that the conditions for performing the centrifugation are severe, the operation for performing the centrifugation is complicated, and besides, the centrifugation takes a long time and requires the use of an expensive centrifuge. Also, there have been problems in conventional methods of lysing the TB bacteria and amplifying or detecting genes of the TB bacteria to be performed subsequent to the collection of the TB bacteria. Examples of conventional lysis methods include chemical methods using organic solvents or the like and physical methods using ultrasonic waves or repeating freezing and thawing. However, since TB bacteria have a high cell-wall lipid content, such conventional lysis methods cannot extract genes sufficiently. In order to achieve sufficient gene extraction, it is necessary to make the treatment conditions more severe, which requires the use of a special device and/or a special reagent. Besides, this may result in a longer treatment period and a more complicated operation. Such problems with regard to the collection, lysis, gene amplification, and the like relate to the entire acid-fast bacteria group including TB bacteria. These problems may occur in the case of other bacteria and viruses. Also, similar problems occur in the case of cells such as leukocytes.

On the other hand, in order to measure the concentration of legionella bacteria, which recently are perceived as a problem, present in baths, swimming pools, and the like or the concentration of Escherichia coli or the like present in rivers, lakes, seas, and the like, it is necessary to collect several liters to several tons of test sample and collect microorganisms or the like from this test sample. However, it is difficult to achieve efficient collection by conventional methods, and the development of a technique enabling the efficient collection of bacteria or the like from a large amount of sample has been desired.

### DISCLOSURE OF INVENTION

Therefore, with the foregoing in mind, it is a first object of the present invention to provide a method of collecting microorganisms or cells easily and in a short time without using any special device, and it is a second object of the present invention to provide a method of specifically amplifying or detecting a gene, by which microorganisms or cells can be detected easily and in a short time without using any special device or special reagent.

In order to achieve the first object, the present invention provides a method of collecting a microorganism or a cell from a liquid sample, including bringing the liquid sample into contact with water-absorbing resin so that a liquid phase part of the liquid sample is absorbed by the water-absorbing resin and the microorganism or the cell is caught on a surface of the water-absorbing resin.

According to the method of the present invention, the microorganism or the cell can be caught by the water-absorbing resin easily in a short time. Thus, centrifugation that requires severe treatment conditions need not be performed. Moreover, it is possible to design the water-absorbing resin to have a required size, amount, or the like in accordance with the amount of test sample. As a result, even when the amount of the test sample is as great as several liters to several tons, target bacteria (e.g., legionella bacteria or Escherichia coli) or the like can be collected merely by bringing the test sample into contact with the water-absorbing resin.

Next, in order to achieve the second object, the present invention provides a method of amplifying or detecting specifically a gene of a microorganism or a cell, including: collecting a microorganism or a cell by the method of the present invention; extracting a gene of the microorganism or the cell by adding an extraction reagent solution containing a nonionic detergent to the microorganism or the cell and heating the resultant mixture; and amplifying or detecting specifically the extracted gene.

This method employs the collection method of the present invention. According to this method, gene extraction can be achieved merely by adding the extraction reagent solution containing the nonionic detergent to the microorganism or the cell and heating the resultant mixture. This allows a quick shift to the subsequent operation such as gene amplification.

### BRIEF DESCRIPTION OF DRAWINGS

FIG. 1 is a cross-sectional view showing an example of the configuration of a centrifugation tube to be used in a collection method according to the present invention.

FIGS. 2A to 2D illustrate major processes of an example of a collection method according to the present invention.

### BEST MODE FOR CARRYING OUT THE INVENTION

Hereinafter, the present invention will be described more specifically.

In the method of collecting a microorganism or a cell according to the present invention, it is preferable that, after the liquid sample has been brought into contact with the water-absorbing resin, a collecting solution further is brought into contact with the water-absorbing resin to collect the microorganism or the cell caught on the surface of the water-absorbing resin in the collecting solution. By repeating this collecting operation a plurality of times, the amount of the collecting solution added decreases gradually, thereby increasing the concentration of the microorganism or the cell in the collecting solution. This allows a subsequent process such as centrifugation to be performed more efficiently. The collecting solution is not particularly limited, and may be, for example, water, distilled water, ion exchanged water, ultrapure water, a buffer, or the like. However, it is preferable that an extraction solution for gene extraction of the microorganism or the cell is used as the collecting solution, as will be described later.

The amount of the liquid sample added preferably is not greater than a water-absorbing capacity of the water-absorbing resin. Also, it is possible to change the amount of the water-absorbing resin in accordance with the amount of the liquid sample to be added so that the amount of the liquid sample to be added would not be greater than a water-absorbing capacity of the water-absorbing resin. Furthermore, the amount of the collecting solution added preferably is greater than a water-absorbing capacity of the water-absorbing resin that has absorbed the liquid phase part of the liquid sample. It is preferable that the water-absorbing resin has a water-absorbing capacity of, for example, 2 to 10000 times its own weight, preferably 5 to 5000 times its own weight, and more preferably 10 to 1000 times its own weight. When treating a large amount of test sample, the treatment preferably is performed using water-absorbing resin with a large water-absorbing capacity, e.g., the one with a water-absorbing capacity of 10000 times its own weight. The water-absorbing resin is not particularly limited, and may be, for example, a hydrophilic cross-linked polymer having a hydrophilic functional group. Examples of the hydrophilic functional group include anionic, nonionic, and cationic hydrophilic functional groups, such as a carboxyl group, a sulfonyl group, and an amino group. Specific examples of the water-absorbing resin include crosslinking substances of carboxymethyl cellulose, hydrolysates of starch-acrylonitrile graft polymer, neutralized substances of starch-acrylic acid graft polymer, crosslinking substances of polyacrylate polymer, crosslinking substances of polymethacrylate polymer, saponified substances of vinyl acetate-acrylic ester copolymer, hydrolysates of acrylonitrile polymer or acrylamide copolymer or crosslinking substances thereof, crosslinking substances of polymers containing a sulfone group, and crosslinking substances of polyethylene oxide or polyethyleneimine. They may be used either alone or in combinations of at least two kinds thereof. Among these, crosslinking substances of partially neutralized polyacrylate or polyacrylic acid are preferable, and polyacrylate is more preferable. Specific examples of polyacrylate include sodium polyacrylate. The form of the water-absorbing resin is not particularly limited, but it is preferable that the water-absorbing resin is provided as particles, as will be described later.

In the method of collecting a microorganism or a cell according to the present invention, it is preferable that a centrifugation tube including a filter that divides an inner space of the centrifugation tube into an upper part and a lower part and water-absorbing resin particles disposed on the filter is provided, the liquid sample is poured into the centrifugation tube to bring the liquid sample into contact with the water-absorbing resin particles, the collecting solution is then poured into the centrifugation tube to bring the collecting solution into contact with the water-absorbing resin particles, and the centrifugation tube is centrifuged so that the collecting solution passes through the filter to move toward a bottom of the centrifugation tube.

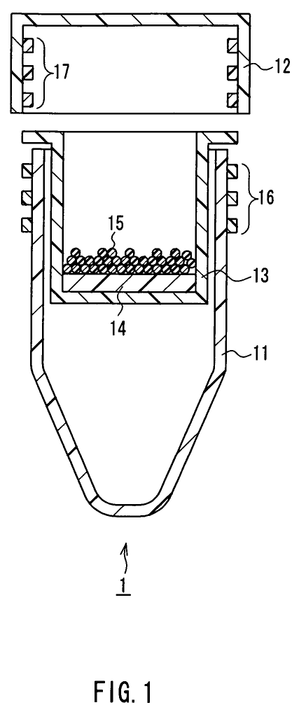

The centrifugation tube used in the above-described method is a centrifugation tube including a filter that divides an inner space of the tube into an upper part and a lower part and water-absorbing resin particles disposed on the filter. FIG. 1 shows an example of such a centrifugation tube. As shown in FIG. 1, main components of this centrifugation tube are a tube body 11 and a cap 12. A screw thread 16 is formed on an upper part of an outer wall of the tube body 11, while a thread groove 17 is formed on an inner wall of the cap 12. Thus, the tube body 11 and the cap 12 are configured so as to be screwed together. A supporter 13 in the shape of a cylinder with a bottom is inserted into the tube body 11. On the bottom of the supporter 13, a filter 14 is disposed, and water-absorbing resin particles 15 are disposed on this filter 14. The filter is not particularly limited, and the pore size thereof may be, for example, in the range from 10 nm to 100 μm, preferably from 0.1 to 20 μm, and more preferably from 0.1 to 10 μm. The material for the filter is not particularly limited, and may be, for example, polyvinylidene fluoride, cellulose nitrate, hydrophilic polyethersulfone, polytetrafluoroethylene, polycarbonate, polyamide, polysulfone, polyethylene, polypropylene, polymer alloy, acetylcellulose, or the like. Among these, polyvinylidene fluoride and polycarbonate are preferable, and polycarbonate is more preferable.

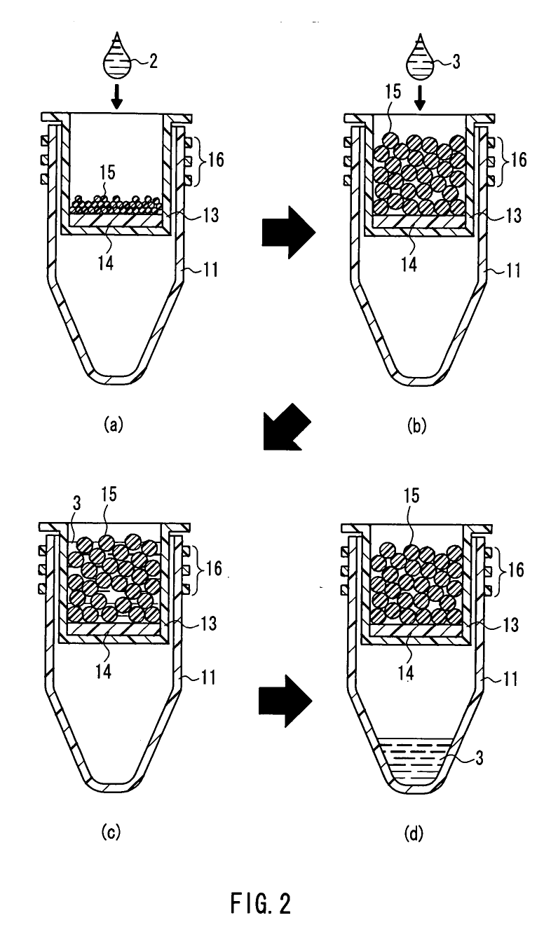

FIGS. 2A to 2D illustrate an example of a method of collecting microorganisms or cells using this centrifugation tube 1. It is to be noted that portions in common between FIG. 1 and FIGS. 2A to 2D are numbered identically. First, as shown in FIG. 2A, a liquid sample 2 is dropped into a tube body 11. Water-absorbing resin particles 15 then swell by absorbing a liquid phase part of the liquid sample 2, as shown in FIG. 2B. The amount of the liquid sample 2 dropped at this time is not greater than the water-absorbing capacity of the water-absorbing resin particles 15 as a whole, and microorganisms or cells adhere to surfaces of the water-absorbing resin particles 15. Thereafter, as shown in FIG. 2B, a collecting solution 3 further is dropped onto the water-absorbing resin particles 15. As described above, it is preferable to use an extraction reagent solution as the collecting solution. The amount of the collecting solution 3 dropped is greater than the water-absorbing capacity of the water-absorbing resin particles 15 that have absorbed the liquid phase part of the liquid sample 2 as a whole. Thus, as shown in FIG. 2C, the collecting solution 3 that has not been absorbed by the water-absorbing resin particles 15 accumulates in a supporter 13, and the microorganisms or the cells are collected in the accumulating collecting solution 3. At this time, in order to improve the collection efficiency of the microorganisms or the cells, it is preferable to cause the water-absorbing resin particles 15 to shake well in the collecting solution 3 by, for example, repeated pipetting. Thereafter, the tube body 11 is capped with a cap 12 (not shown) and then is centrifuged. In the present invention, the conditions for the centrifugation are not particularly limited. For example, the centrifugation may be performed at 500 to 13000 g for 3 seconds to 60 minutes, preferably at 1000 to 10000 g for 10 seconds to 10 minutes, and more preferably at 5000 g for 1 minute. The centrifuge to be used is not particularly limited, and may be, for example, a desktop centrifuge. After the centrifugation, the collecting solution accumulates in the lower part of the centrifugation tube, as shown in FIG. 2D. This collecting solution is collected and used as a sample in a genetic analysis that will be described later.

Next, in the method of amplying or detecting a gene according to the present invention, the extraction reagent solution preferably is used as the collecting solution in the above-described method of collecting microorganisms or cells. When the collecting solution is the extraction reagent solution, the gene can be extracted by heating the collecting solution simply as it is. This allows the operation for adding an extraction reagent solution to be omitted. The heating temperature of the extraction reagent solution is not particularly limited, but preferably is not lower than 70° C. and lower than 100° C. By setting the heating temperature to a temperature lower than 100° C., the following advantages can be obtained, for example: there is reduced concern that a sample might be scattered, and the temperature can be controlled easily so that a special heater is not required. The heating temperature preferably is not lower than 80° C. and lower than 100° C., and most suitably 96° C. Furthermore, the heating is performed, for example, for 1 to 30 minutes, preferably for 10 minutes. The pH of the liquid is, for example, in the range from 7.0 to 12.0, preferably 8.0. The concentration of the nonionic detergent in the extraction reagent solution is, for example, 0.01 to 10 wt %, preferably 0.5 to 2.0 wt %, and more preferably 1.0 wt %.

Examples of the nonionic detergent include: D-sorbitol fatty acid esters such as Span 20, Span 40, Span 60, Span 65, Span 80, and Span 85 (all manufactured by Nacalai Tesque, Inc., for example); polyoxyethyleneglycol sorbitan alkyl esters such as Tween 20, Tween 21, Tween 40, Tween 60, Tween 65, Tween 80, Tween 81, and Tween 85 (all manufactured by Nacalai Tesque, Inc., for example); polyoxyethyleneglycol p-t-octylphenyl ethers such as Triton X-100 (manufactured by Nacalai Tesque, Inc., for example). These detergents may be used either alone or in combinations of at least two kinds thereof. Among these, Triton X-100, Tween 20, and Tween 21 are preferable, and Triton X- 100 is more preferable.

In the method of amplifying or detecting a gene according to the present invention, it is preferable that the extraction reagent solution further contains a metal chelating agent. The metal chelating agent serves to prevent a gene-degrading enzyme such as DNase contained in a sample from degrading the gene, for example. The concentration of the metal chelating agent in the extraction reagent solution is, for example, 0.1 to 100 mM, preferably 1.0 mM. Examples of the metal chelating agent include ethylenediaminetetraacetic acid (EDTA), ethylene glycol bis(β-aminoethyl ether)-N,N,N′,N′-tetraacetic acid (EGTA), diaminocyclohexane tetraacetic acid, o-phenanthroline, and salicylic acid. These metal chelating agents may be used either alone or in combinations of at least two kinds thereof. Among these, EDTA and EGTA are preferable, and EDTA is more preferable.

The microorganism to which the present invention is applicable is not particularly limited. Examples thereof include acid-fast bacteria, atypical mycobacteria, gonococcus, legionella bacteria, mycoplasmas, spirochetes, syphilis spirochetes, chlamydiae, rickettsiae, Mycobacterium leprae, Spirillum minus, staphylococci, streptococci, Escherichia coli, Pseudomonas aeruginosa, Pasteurella pestis, viruses, Japanese encephalitis virus, hepatitis B virus, hepatitis C virus, ATLV, HIV, and Ebola virus. Examples of the acid-fast bacteria include M. avium, M. intracellularae, M. gordonae, M. tuberculosis, M. kansasii, M. fortuitum, M. chelonae, M. bovis, M. scrofulaceum, M. paratuberculosis, M. phlei, M. marinum, M. simiae, M. scrofulaceum, M. szulgai, M. leprae, M. xenopi, M. ulcerans, M. lepraemurium, M. flavescens, M. terrae, M. nonchromogenicum, M. malmoense, M. asiaticum, M. vaccae, M. gastri, M. triviale, M. haemophilum, M. africanum, M. thermoresistable, and M. smegmatis. Furthermore, the cell to which the present invention is applicable is not particularly limited. Examples thereof include cellular tissues containing a genetic material, such as human leukocytes, human somatic cells, or cells of other animals and plants.

In the present invention, the liquid sample may be, for example, sputum, spinal fluid, feces, saliva, blood, a tissue, urine, or a sample obtained by pretreating any one of these biological samples. Examples of the pretreated sample include a sample obtained by treating sputum with N-acetyl-L-cysteine (NALC) and sodium hydroxide (NaOH). Other than these, water in baths, water in swimming pools, water in fish farms, water in rivers, lake water, seawater, and the like also may be used as the liquid sample in the present invention.

The amount of the liquid sample in the present invention is not particularly limited. However, when a relatively small amount of sample is required as in the case of a clinical test or the like, the amount of the liquid sample is, for example, in the range from 5 μL to 10 mL, preferably from 10 μL to 1 mL, and more preferably from 50 μL to 500 μL. On the other hand, when a relatively large amount of sample is used, for example, as in the case where the concentration of microorganisms or the like in water in a bath, a river, or the like is measured as part of water examination, the amount of the liquid sample should be within the range determined by ministerial ordinances given by the respective government agencies, for example. More specifically, the amount of the liquid sample is in the range from 1 mL to 10 L, preferably from 10 mL to 1 L, and more preferably from 50 mL to 200 mL. Examples of the ministerial ordinances include “Manual of Prevention Measures against Legionella Infection in Circulating Type Bath” (issued by the director of the Environmental Health Division of the Health Service Bureau in the Ministry of Health, Labour and Welfare in Japan as a 95th notification dated Sep. 11, 2001). The manual describes, for example, legionella bacteria in a bath should be less than 10 CFU per 100 mL. Thus, when detecting legionella bacteria in a bath, it is necessary to use at least 100 mL of sample. It is to be noted here that the amount of the liquid sample preferably does not exceed the water-absorbing capacity of the water-absorbing resin, as described above.

Next, the method of amplifying or detecting a gene according to the present invention can be carried out in the following manner, for example. First, to a buffer having a pH in the above-described predetermined range, a metal chelating agent such as EDTA is added as necessary and a nonionic detergent further is added, thus preparing an extraction reagent solution. As the buffer, Tris-HCl buffer, HEPES buffer, MOPS buffer, HEPPS buffer, TAPS buffer, phosphate buffer, or the like may be used. This extraction reagent solution preferably is sterilized by high-pressure steam in an autoclave. On the other hand, a sample solution is prepared. For example, a sputum specimen is homogenized and sterilized by the N-acetyl-L-cysteine-NaOH method (NALC-NaOH method) or the like. The thus-treated specimen (a liquid sample) is added to a centrifugation tube (e.g., the one shown in FIG. 1) that includes a collection filter inside and water-absorbing resin particles disposed on the collection filter, and the extraction reagent solution (a collecting solution) further is added to the centrifugation tube. The centrifugation tube is then centrifuged (e.g., 5000 g, 1 minute) so that the extraction reagent solution accumulates in the lower part of the centrifugation tube. An extraction treatment is then performed by heating the extraction reagent solution that has accumulated, either as it is or after being transferred into a different tube, at a temperature in the above-described predetermined range using a heating block or the like. Examples of heating means other than the heating block include a water bath, a microwave oven, and an air bath. The specimen that has subjected to the above-described extraction treatment may be subjected to a gene amplification treatment or a gene detection treatment simply as it is or after being pretreated. Examples of the method of performing the gene amplification or gene detection include a PCR method and modifications of the PCR method, such as a RT-PCR method. Furthermore, examples of the gene to be analyzed include DNA and RNA. In the case where the collecting solution is different from the extraction reagent solution, the extraction reagent solution is added to the collecting solution to carry out the extraction treatment.

### EXAMPLES

A genetic analysis was carried out with regard to cultures of TB bacteria in the following manner.

Preparation of TB Bacteria

A clinical isolate of TB bacteria was cultured in a product named MycoBroth (Kyokuto Pharmaceutical Industrial Co., Ltd.) at 35° C. until a turbidity corresponding to #1 of the McFarland turbidity standard was obtained. Then, the culture was diluted with 0.067 M phosphate buffer (pH: 6.8) so as to achieve a series of 10-fold dilutions (10⁰-fold to 10¹⁰-fold). The thus-obtained solutions were used as samples.

Operation For Collecting TB Bacteria

Centrifugation tubes (see FIG. 1), each including a filter unit (see FIG. 1) provided with a product named Isopore Filter (pore size 3 μm, Millipore Corporation) and 0.0065 g of water-absorbing resin particles (product name “AQUA KEEP 10SH P”, SUMITOMO SEIKA CHEMICALS CO., LTD.) disposed on this filter unit, were provided. 100 μL of the above-described samples with the diluted TB bacteria were added to these centrifugation tubes, respectively. Thereafter, 500 μL of an extraction reagent solution further was added to each of the centrifugation tubes. This extraction reagent solution also served as a collecting solution and had been prepared by dissolving Triton X-100 (Nacalai Tesque, Inc.) in TE buffer (10 mM EDTA and 25 mM Tris-HCl, pH 8.0) so that its concentration became 1% and then autoclaving the mixture. The centrifugation tubes were then centrifuged at 5000 g for 1 minute, and the filtrates (about 100 μL) that accumulated in the bottom of the centrifugation tubes were collected.

Gene Extraction

The filtrates were heated at 96° C. for 10 minutes in a heating block to lyse the TB bacteria.

Gene Detection By PCR

To 25 μL of each of the lysates, 50 μL of the master mixture of a product named AMPLICOR Amplification Kit (Nippon Roche K.K.) and 25 μL of 15 mM magnesium acetate were added to cause a PCR. Conditions and an operation for causing the PCR were pursuant to the document attached to the kit. Detection of a gene was carried out using a product named AMPLICOR Detection Kit (Nippon Roche K.K.). The operation for detecting the gene was carried out pursuant to the document attached to the kit.

### Comparative Example 1

As a comparative example, samples prepared in the same manner as in the above were treated using a product named AMPLICOR Pretreatment Kit (Nippon Roche K.K.). With regard to the thus-treated samples, gene detection by the PCR was carried out in the same manner as in the above.

Results

In Example 1 and Comparative Example 1, the samples with a dilution factor of up to 10⁵ were judged as tuberculosis positive and those with a dilution factor of greater than 10⁵ were judged as tuberculosis negative. Furthermore, internal controls (ICs) were all judged as tuberculosis positive in both Example 1 and Comparative Example 1. Thus, it can be said that the effect of collecting and lysing the cultures of the TB bacteria achieved by the method of Example 1 of the present invention is equivalent to that achieved by the conventional method of Comparative Example 1. Moreover, from the fact that ICs were all judged as tuberculosis positive, it is understood that contamination of substances that interfere with the PCR was also suppressed. Besides, according to the method of Example 1 of the present invention, the time required for the collection and the lysis was reduced to about ⅕ of that in the method of Comparative Example 1.

### Example 2

Sputum specimens (7 specimens) collected from patients with suspected tuberculosis (TB) were treated by the NALC-NaOH method, thus preparing samples. Then, with regard to these samples, collection, gene extraction, and gene detection by the PCR were carried out in the same manner as in Example 1. The results are shown in Table 1 below.

### Comparative Example 2

Samples prepared in the same manner as in Example 2 were treated using a product named AMPLICOR Pretreatment Kit (Nippon Roche K.K.). With regard to the thus-treated samples, gene detection by the PCR was carried out in the same manner as in Example 1. The results are shown in Table 1 below.

TABLE 1

| Sample No. | Example* | Comparative Example* |
|---|---|---|
| 1 | 1.786 | 2.796 |
| 2 | 2.809 | 2.662 |
| 3 | 2.875 | 2.85 |
| 4 | 2.668 | 2.8 |
| 5 | 2.772 | 2.816 |
| 6 | 2.752 | 2.741 |
| 7 | 2.728 | 2.736 |

*absorbance (wavelength: 450 nm)

As shown in Table 1, in both Example 2 and Comparative Example 2, all the 7 specimens were judged as tuberculosis positive, and internal controls (ICs) also were all judged as tuberculosis positive. Thus, it can be said that the method of the present invention can exhibit a similar performance to that of the conventional method. Moreover, the method of the present invention can perform the operations for collecting and lysing the TB bacteria more easily and more quickly than the conventional method.

### INDUSTRIAL APPLICABILITY

As specifically described above, the present invention provides a method of collecting microorganisms or cells easily and in a short time without using any special device. Therefore, by applying the method of the present invention to, for example, a pretreatment of a sample to be analyzed in an examination of microorganisms or the like utilizing gene amplification and detection, higher efficiency of the examination can be realized easily.

1. A method of collecting a microorganism or a cell from a liquid sample, comprising:
bringing the liquid sample into contact with water-absorbing resin so that a liquid phase part of the liquid sample is absorbed by the water-absorbing resin and the microorganism or the cell is caught on a surface of the water-absorbing resin.

2. The method according to claim 1, wherein after the liquid sample has been brought into contact with the water-absorbing resin, a collecting solution further is brought into contact with the water-absorbing resin to collect the microorganism or the cell caught on the surface of the water-absorbing resin in the collecting solution.

3. The method according to claim 2, wherein a centrifugation tube comprising a filter that divides an inner space of the tube into an upper part and a lower part and water-absorbing resin particles disposed on the filter is provided, the liquid sample is poured into the centrifugation tube to bring the liquid sample into contact with the water-absorbing resin particles, the collecting solution is then poured into the centrifugation tube to bring the collecting solution into contact with the water-absorbing resin particles, and the centrifugation tube is centrifuged so that the collecting solution passes through the filter to move toward a bottom of the centrifugation tube.

4. The method according to claim 3, wherein the centrifugation is performed at 500 to 13000 g for 3 seconds to 60 minutes.

5. The method according to claim 1, wherein an amount of the liquid sample added is not greater than a water-absorbing capacity of the water-absorbing resin.

6. The method according to claim 2, wherein the amount of the collecting solution added is greater than a water-absorbing capacity of the water-absorbing resin that has absorbed the liquid phase part of the liquid sample.

7. The method according to claim 1, wherein the water-absorbing resin is a hydrophilic cross-linked polymer having a hydrophilic functional group.

8. The method according to claim 1, wherein the microorganism to be collected is at least one selected from the group consisting of acid-fast bacteria, atypical mycobacteria, gonococcus, legionella bacteria, mycoplasmas, spirochetes, syphilis spirochetes, chlamydiae, rickettsiae, Mycobacterium leprae, Spirillum minus, staphylococci, streptococci, Escherichia coli, Pseudomonas aeruginosa, Pasteurella pestis, viruses, Japanese encephalitis virus, hepatitis B virus, hepatitis C virus, ATLV, HIV, and Ebola virus.

9. The method according to claim 8, wherein the acid-fast bacterium is at least one selected from the group consisting of M. avium, M. intracellularae, M. gordonae, M. tuberculosis, M. kansasii, M. fortuitum, M. chelonae, M. bovis, M. scrofulaceum, M. paratuberculosis, M. phlei, M. marinum, M. simiae, M. scrofulaceum, M. szulgai, M. leprae, M. xenopi, M. ulcerans, M. lepraemurium, M. flavescens, M. terrae, M. nonchromogenicum, M. malmoense, M. asiaticum, M. vaccae, M. gastri, M. triviale, M. haemophilum, M. africanum, M. thermoresistable, and M. smegmatis.

10. The method according to claim 1, wherein the liquid sample is at least one selected from the group consisting of sputum, spinal fluid, feces, saliva, blood, tissues, swab, liquid obtained by gastrolavage, urine, samples obtained by pretreating these biological samples, water in baths, water in swimming pools, water in fish farms, water in rivers, lake water, and seawater.

11. The method according to claim 1, wherein the amount of the liquid sample is in a range from 50 μL to 500 μL.

12. The method according to claim 1, wherein the amount of the liquid sample is in a range from 50 mL to 200 mL.

13. An implement for collecting a microorganism or a cell comprising a centrifugation tube,
the centrifugation tube comprising:
a filter that divides an inner space of the centrifugation tube into an upper part and a lower part; and
water-absorbing resin particles disposed on the filter.

14. A method of amplifying or detecting specifically a gene of a microorganism or a cell, comprising:
collecting a microorganism or a cell by the method according to claim 1;
extracting a gene of the microorganism or the cell by adding an extraction reagent solution containing a nonionic detergent to the microorganism or the cell and heating the resultant mixture; and
amplifying or detecting specifically the extracted gene.

15. The method according to claim 14, wherein the extraction reagent solution also serves as the collecting solution.

16. The method according to claim 14, wherein the heating temperature is not lower than 70° C. and lower than 100° C.

17. The method according to claim 14, wherein the heating is performed for 1 to 30 minutes.

18. The method according to claim 14, wherein the heating is performed at 96° C. for 10 minutes.

19. The method according to claim 14, wherein a pH of the extraction reagent solution is in a range from 7.0 to 12.0.

20. The method according to claim 14, wherein a concentration of the nonionic detergent in the extraction reagent solution is in a range from 0.01 to 10 wt %.

21. The method according to claim 14, wherein the nonionic detergent is at least one selected from the group consisting of D-sorbitol fatty acid esters, polyoxyethyleneglycol sorbitan alkyl esters, and polyoxyethyleneglycol p-t-octylphenyl ethers.

22. The method according to claim 14, wherein the extraction reagent solution further contains a metal chelating agent.

23. The method according to claim 22, wherein a concentration of the metal chelating agent in the extraction reagent solution is 0.1 to 100 mM.

24. The method according to claim 22, wherein the metal chelating agent is at least one selected from the group consisting of ethylenediaminetetraacetic acid (EDTA), ethylene glycol bis(β-aminoethyl ether)-N,N,N′,N′-tetraacetic acid (EGTA), diaminocyclohexane tetraacetic acid, o-phenanthroline, and salicylic acid.

25. The method according to claim 14, wherein the gene is amplified or detected specifically by a polymerase chain reaction (PCR) method.
